(19) United States
(12) Patent Application Publication
(10) Pub. No.: US 2016/0158099 A1
(43) Pub. Date: Jun. 9, 2016

(54) ELBOW DUAL COMPRESSION ARMBAND WITH INTERCHANGEABLE COMPRESSION PLATFORM

(71) Applicant: Scott Kupferman, North Babylon, NY (US)

(72) Inventor: Scott Kupferman, North Babylon, NY (US)

(21) Appl. No.: 14/936,600

(22) Filed: Nov. 9, 2015

Related U.S. Application Data
(60) Provisional application No. 62/077,889, filed on Nov. 10, 2014.

(51) Int. Cl. A61H 39/04; A61F 7/08

(52) U.S. Cl. CPC A61H 39/04; A61F 7/08; A61H 2201/165; A61H 2201/10; A61H 2201/1645; A61F 2007/0032

(57) ABSTRACT

Apparatus for applying pressure, heat, cold or accu-pressure to a limb, comprises band members, at least a portion of which are elastic. A tab element, at least a portion of which is stiff and plastic, forms a catch for receiving and holding various treatment modalities. A shield, at least a portion of which is thin, flexible and smooth, prevents the user from becoming uncomfortable during treatment. A safety tab, at least a portion of which is stiff and plastic prevents the apparatus from coming undone, and thereby facilitates securement of the armband around the affected area. A pocket element receives various treatment modalities. A rectangular frame, at least a portion of which is stiff and plastic, connects band members and forms a cuff-like apparatus.

### CROSS REFERENCE TO RELATED APPLICATIONS

This application claims the priority of U.S. Provisional Patent Application No. 62/077,889, entitled Improved Elbow Dual Compression Armband With Interchangeable Compression Platform filed Nov. 9, 2014.

### TECHNICAL FIELD

The invention relates to an apparatus and method for the prevention, treatment and healing strain and stress injuries of the muscles and tendons surrounding the elbow, such as lateral epicondylitis (also known as tennis elbow) and medial epicondylitis (also known as golfer's elbow) in humans.

### STATEMENT REGARDING FEDERALLY SPONSORED RESEARCH OR DEVELOPMENT

(Not applicable)

### BACKGROUND OF THE INVENTION

In the past, a great deal of work has been devoted to the development of a therapeutic treatment having as its object the prevention and/or treatment of injury to the tissues comprising and surrounding the elbow and, in particular, providing relief from the pain and discomfort of epicondylitis.

Epicondylitis is a painful and sometimes disabling inflammation of the muscle and surrounding tissues of the elbow caused by repeated stress and strain on the forearm near the medial or lateral epicondyle of the humerus (arm bone).

Epicondylitis is a common and often painful condition that is caused by an inflammation on the part of the elbow joint near the bony protrusions. Micro tears and lack of adequate blood supply in the tendon origin caused by heavy or repetitive activity are what commonly aggravate elbow conditions, such as tennis and golfers elbow. Most cases are curable without surgical intervention but the duration of tennis elbow is often over 1 year with many setbacks along the way.

Currently, there are various treatments for epicondylitis, but they do not target the root of the problem—lack of blood flow impeding tendon healing—head on. Muscles act as pumps to drive blood flow delivering vital nutrients such as vitamins, minerals, amino acids and oxygen, so it is important to stimulate them, as they are needed for optimal performance and healing throughout the body. The key reason tennis elbow lasts for so long with numerous painful setbacks along the way is the lack of adequate blood supply.

One such treatment for epicondyltis has been achieved by a clasp or brace, such as the one disclosed in U.S. Pat. No. 6,007,508. This invention discloses an epicondylitis clasp, which introduces pressure in an adjustable fashion onto the epicondyle. According to the invention, this is brought about by virtue of the fact that arranged at the one end of the brace on the inside of the clasp is a pad which serves to exert pressure on the epicondyle. Athletes also commonly use elastic sports tape “bandages” such as those sold under the trademark Ace.

Another band approach to treatment is disclosed in U.S. Pat. No. 4,763,901. That patent discloses a pair of bands positioned at opposite sides of the elbow and connected by an elastic band to provide resistance during exercise.

Still, another similar device is disclosed in E.P. Pat. No. 2,184,040. The band disclosed in this patent provides a pad on its inner side perhaps for the purpose of exerting pressure on the affected area of the arm so that pressure is achieved. This patent discloses a device similar to U.S. Pat. No. 6,007,508 and C.N. Pat. No. 101,732,120 in that they are all designed for the purpose of providing a pressure-exerting device, specifically a pad, to the necessary area for therapeutic treatment. However, they all suffer from the disadvantage that they do not otherwise address the issue of tendon healing and are not easily used.

### SUMMARY OF THE INVENTION

Various other treatments for epicondylitis have been proposed in the art. Applying heat and ice in combination is a common treatment as ice controls swelling and heat heals and promotes blood flow and also relieves tightness and pain. A number of alternative therapies are available as well. For example, acupuncture has also been tried in this field to relieve the symptoms of epicondylitis. There still remains, however, the need to devise a comprehensive rehabilitation system for those suffering from epicondylitis that accelerates tendon healing by substantially increasing blood flow.

The present invention now realizes a highly effective means of treating epicondylitis through an integrated treatment system, which incorporates different treatment modalities (including, heat therapy, cold therapy, and targeted acupressure therapy) onto one platform, combining it with corrective exercise thereby creating a comprehensive therapy rehabilitation system that substantially increases blood flow and accelerates the epicondyle tendon healing process.

The present invention is directed to an apparatus and method that satisfies the need for a comprehensive rehabilitation system incorporated onto one platform that targets the root cause of epicondylitis by increasing blood flow and accelerating epicondyle tendon healing. The apparatus comprises a tri-faceted compression system that can be adapted to allow the integration of various treatment modalities. The inventive compression system has a versatile compression platform which is capable of receiving modules for supporting and executing the various treatment modalities. The multiple compression system also has an easy smart tab, which prevents the compression strap system from becoming undone. The easy smart tab facilitates mounting and fastening of the apparatus by the user. The dual compression system also contains a breathable compression platform. When the apparatus is completely fastened the interchangeable compression platform is nestled on top of the breathable compression platform. The breathable, expandable compression platform facilitates efficient temperature transfer, which furthers the therapeutic effect of the treatment modalities. Also enables the patient to be more compliant with implementing treatment as it is a hands free treatment system.

The present invention has a number of advantages. In particular, it contains an easy smart tab, which helps the user mount and fasten the apparatus and removes the difficulties inherent in other like armbands. Additionally, it contains an interchangeable compression platform tailored for receiving various treatment modalities, including heat therapy, cold therapy and acupressure therapy, which increase blood flow and accelerate the healing of the epicondyle tendon. Moreover, it contains a breathable and expandable platform, which facilitates efficient temperature transfer to the affected area of the user's arm optimizing the therapeutic effect of the apparatus.

### BRIEF DESCRIPTION OF THE DRAWINGS

The operation of the inventive armband will become apparent from the following description taken in conjunction with the drawings, in which:

FIG. 1 is a side cross-sectional view illustrating an embodiment of the inventive armband in its final fastened state, positioned around the user's arm;

FIG. 2 is also a side cross-section view illustrating an embodiment of the inventive armband in which it is partially fastened, and also in which a treatment modality is shown in place;

FIG. 3 is a side view of an embodiment of the inventive armband in which the armband is completely loosened.

FIG. 4 is a perspective view illustrating an embodiment of the inventive armband in the which the armband is completely loosened with a tab catching the adjusting mechanism and preventing the armband from becoming undone;

FIG. 5 is a perspective view of the inventive armband fastened around the user's arm, which displays the tab element in which the treatment modality is inserted before final fastening of the armband, in the open configuration;

FIG. 6 illustrates one of the various treatment modalities of the inventive armband, namely the spike plate used for targeted acupressure, with its removal finger strap, which allows use without the armband, in place;

FIG. 7 is a perspective view of the inventive armband fastened around the user's arm, which displays the pocket element in which the spike plate treatment modality has been inserted into tab 32 after removal of the fingerstrap;

FIG. 8 is aerial top plan view of pressure-applying band in its unfastened state, illustrating a treatment device secured in place by pockets;

FIG. 9 is a perspective view of the inventive armband fastened around the user's arm, which displays the spike plate securely held in position;

FIG. 10 is a schematic side view of a preferred embodiment of the inventive armband; and

FIG. 11 is a schematic top plan view of a preferred embodiment of the inventive armband.

### DETAILED DESCRIPTION OF THE PREFERRED EMBODIMENTS

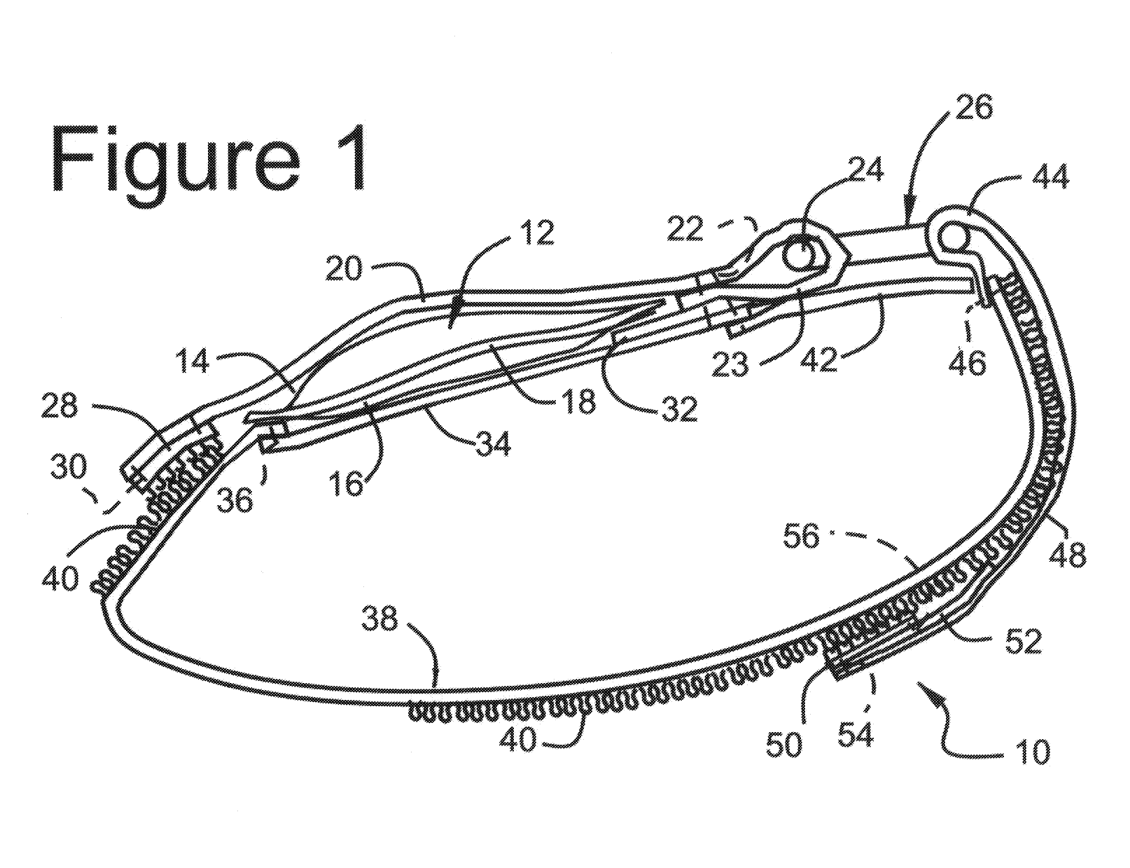

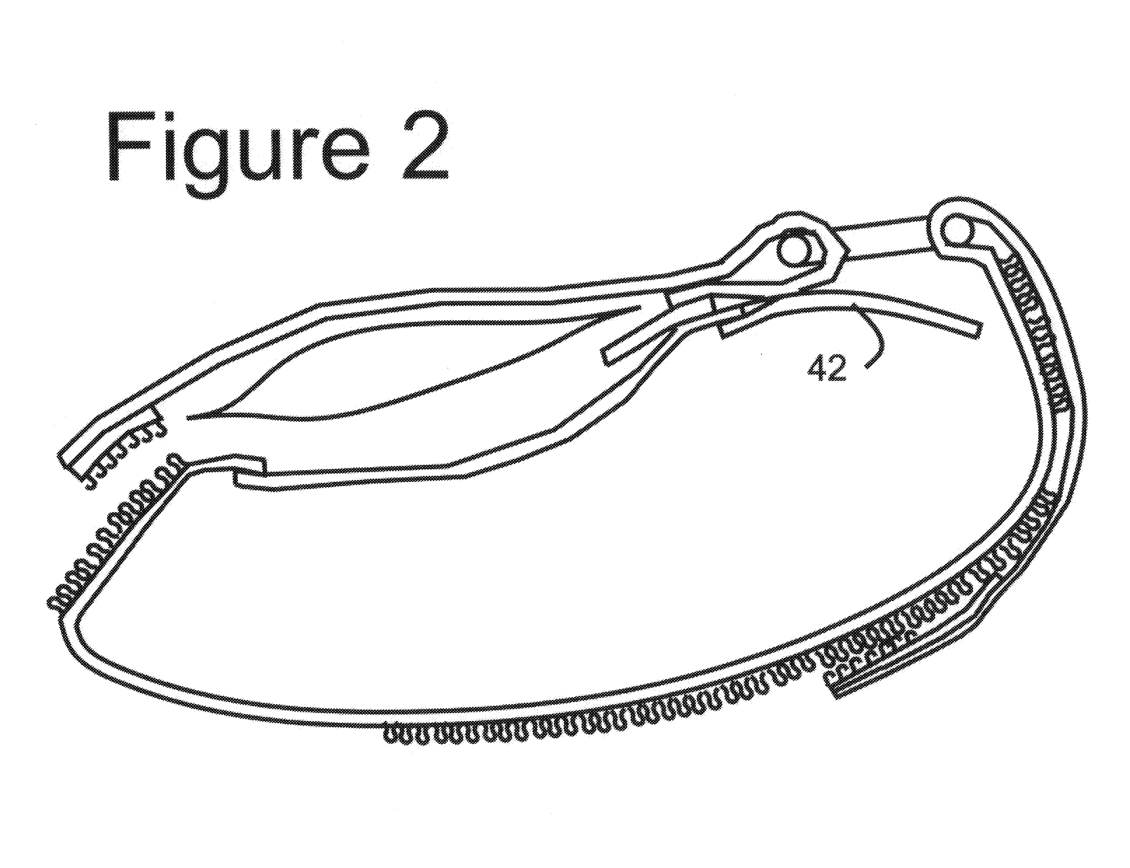

Referring to FIGS. 1-5, an illustrative embodiment of the inventive armband 10 offering the capability of multiple, for example dual, compression functionalities through the use of a plurality, for example two, compression components is illustrated.

The inventive armband 10 has as its primary function the pressurized application of a treatment modality, such as heat. Such pressurized application is achieved by making armband 10, overall, elastic, for example with the inclusion of bands of rubber in the armband components, or making one or more components of a stretchable material

Armband 10 must exert sufficient force to achieve good contact in the case of heat or cold transfer modalities and sufficient force in the case of acupressure modalities, for example pinpoint acupressure modalities. Accordingly, armband 10 is configured to allow the adjustment of pressure and thus accommodate different modalities and different users desired based, for example, on experience with the particular inflammation, pain or the like being treated. The structural means for achieving such variation is detailed below.

Heat may be provided by a heat providing component 12. Heat providing component 12 comprises an outer film member 14 and an inner film member 16 which are heat sealed to each other forming a peripheral heat sealed edge 18. Heat providing component 12 thus provides a sealed compartment for receiving, for example, a liquid material capable of being heated and having a significant heat capacity allowing it to release heat to an area to be treated after being heated, for example, in a cup of boiling water. In accordance with the inention, the heat pack is a reusable instant heat pack of conventional design that resets in boiling water.

Heat providing component 12 is disposed in a compartment formed by an elastic pressure-applying band section 20 which is folded upon itself at one end by stitches 22, to form a loop 23 enclosing one side 24 of a rectangular frame 26. At the other end, a plain fabric fastening element, such as Velcro, carrying, for example, male hook fastening element 28 is secured by stitches 30.

A stiff flexible plastic tab 32 is also secured by stitches 22 to form a catch for receiving and holding heat providing component 12 while armband 10 is being put on by the person using the inventive armband 10. Stitches 22 also secure elastic member 34 to the assembly. Elastic member 34 is made of a thin elastic member in order to promote the transfer of heat, cold or other treatment modality from the treatment member, such as heat providing component 12. In the case of spike pressure application, the elastic must also be flexible enough to allow the points of the spikes to apply point pressure to the affected area. Elastic member 34 is, in turn, secured by stitches 36 to main band member 38.

Main band member 38 holds female, for example hook, mating attachment members 40, which mates with male hooks on fastening element 28, completing the compartment holding heat transfer member 12.

A shield 42 is also secured to the assembly by stitches 22. Shield 22 is made of a thin flexible smooth plastic member which serves to provide a smooth surface under pressure so that loops 44 and 23 to not make the user uncomfortable.

Main band member 38 is secured by stitches 46 to adjustable securement member 48. Adjustable securement member 48 may be positioned as desired to achieve the desired degree of pressure. The end of adjustable securement member 48 is secured to a hook Velcro member 50 and a stiff plastic safety tab 52 by stitches 54. The other end of hook Velcro member 50 is secured by stitches 56 two stiff plastic safety tab 52.

As can be seen in FIG. 1, the treatment component 12 is held in place between plastic tab 32 and the closed end of pressure-applying band section 20 adjacent fastening element 28.

As an alternative to a simple tab, a full pocket may be provided for receiving various epicondylitis treatment modalities, such as a cold pack, a hot pack or a spike plate.

The invention contemplates an expandable compression platform which can accommodate treatment elements of various sizes through the stretching of elastic pressure-applying band section 20 and elastic member 34. The armband 10 also includes a structure providing for adjusting, for example for the purpose of tightening and fastening a treatment modality compression component such as heat applying member 12 around the user's arm.

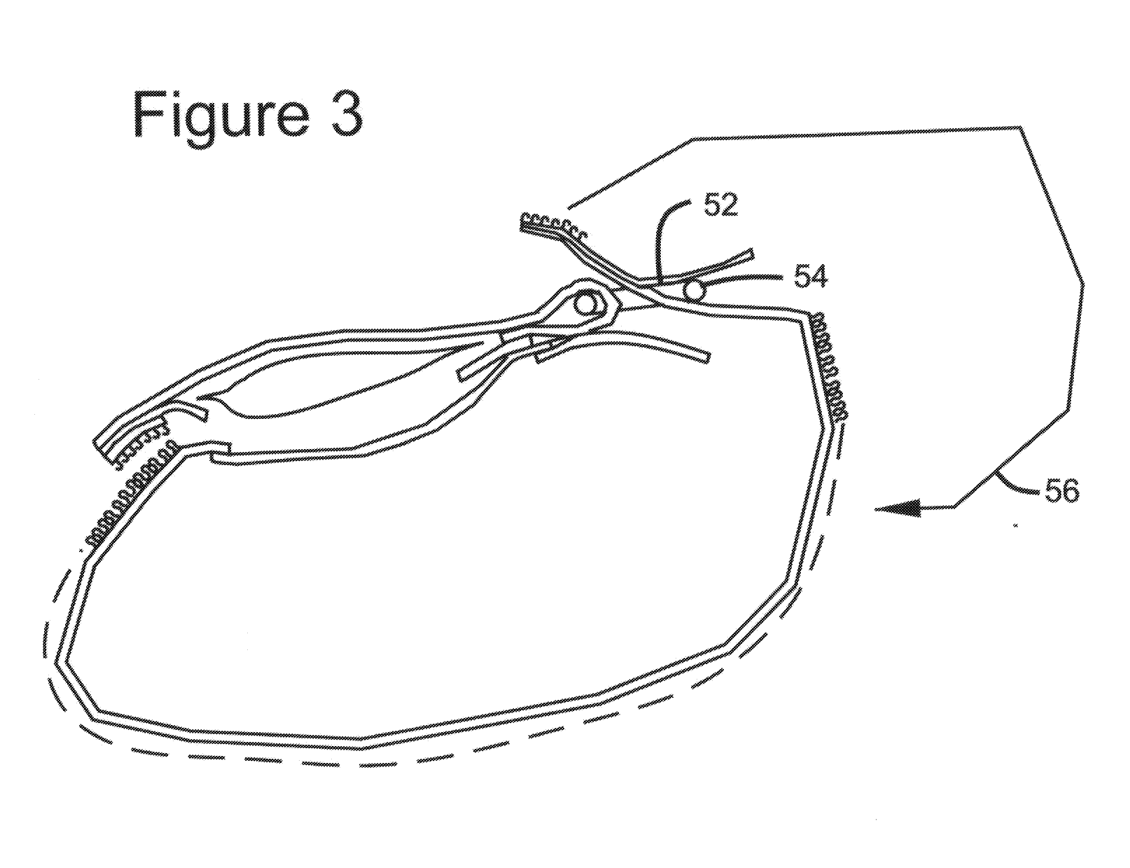

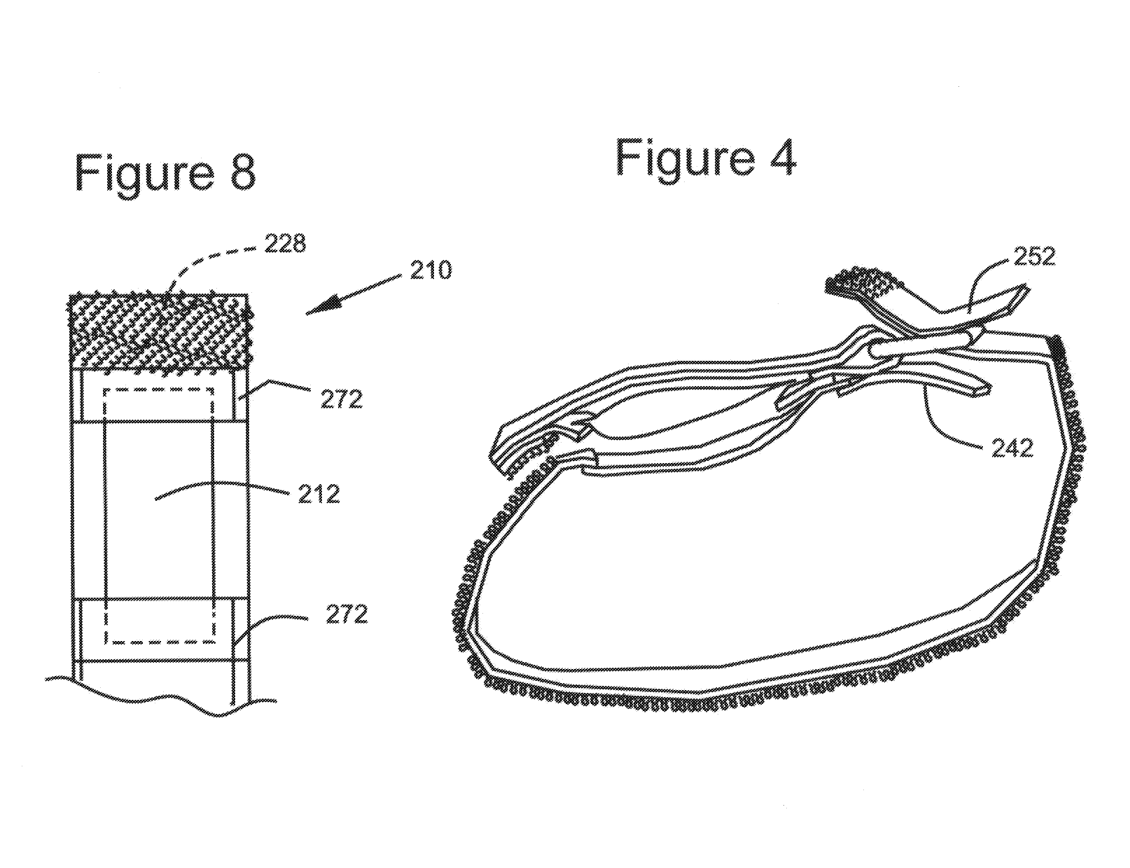

Referring to FIGS. 3 and 4, armband 10 provides for adjusting and tightening the breathable expandable compression platform enclosing treatment device 12 around the affected area of the user's arm. The end of main band member 38 is put through rectangular frame 26. The outward end of plastic safety tab 52 prevents main member 38 from leaving the rectangular frame of the buckle by catching on side 54 of rectangular frame buckle 52, as illustrated in FIG. 3. Adjustable securement member 48 is then folded on itself in the directions indicated by arrow 56, as illustrated in FIG. 3. The end of adjustable securement member 48, after being wrapped around frame 26 and adjusted to the length or pressure desired is secured to hook Velcro member 50 to mating attachment member 40. Result is the formation of the cuff-like armband 10 in FIGS. 1-5.

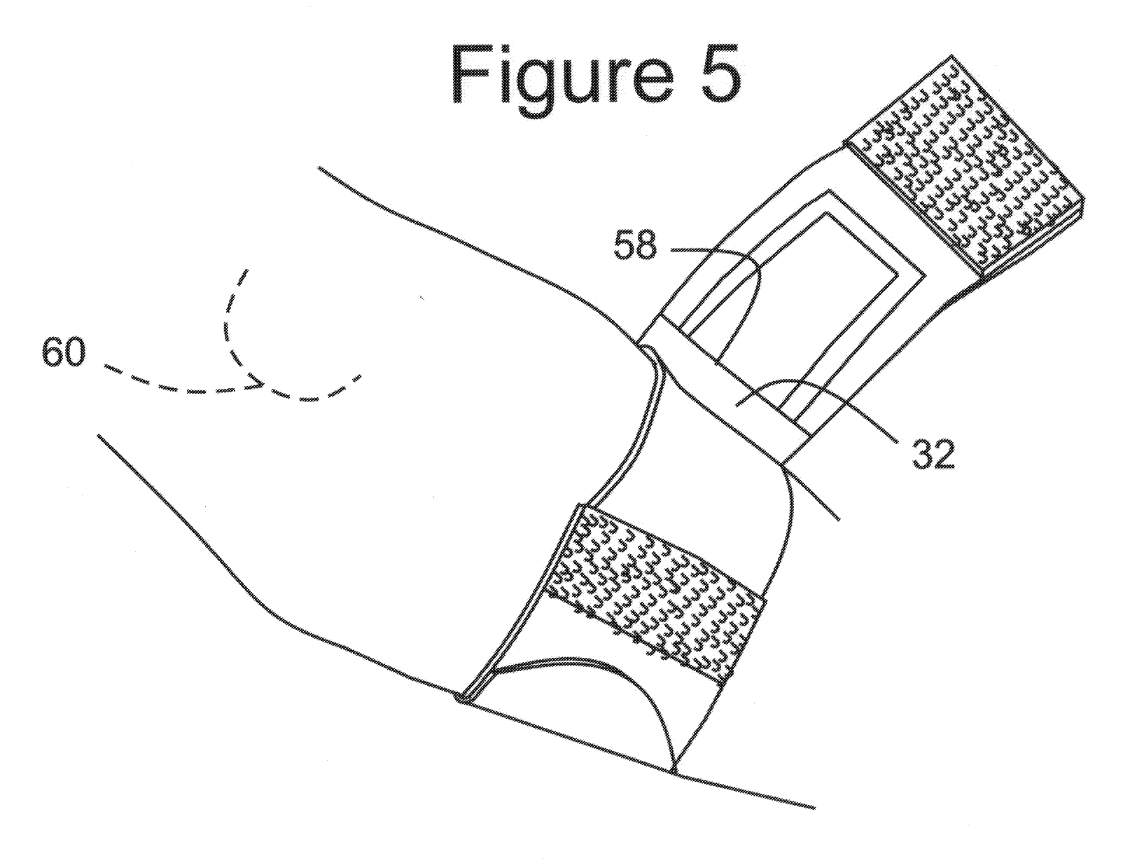

As shown in FIG. 5, flexible plastic tab 32, located on elastic band section 20 and elastic member 34 cooperate to prevent treatment device 12 from slipping off the adjustable armband 10, thus facilitating the fastening of treatment device 12 to armband 10.

In similar fashion, when armband 10 is in an unfastened position safety tab 52 catches rectangular frame 26 of the adjusting mechanism 11, preventing the cuff from coming undone, and thereby facilitating securement of the armband around the affected area, as illustrated in FIG. 3.

A pocket 58 is formed by open tab 32 on the pressure-applying band section 20. In one embodiment of the inventive armband, the pocket 58 is tailored specifically to fit and receive various treatment modalities, including heat providing component 12, cold therapy and acupressure therapy.

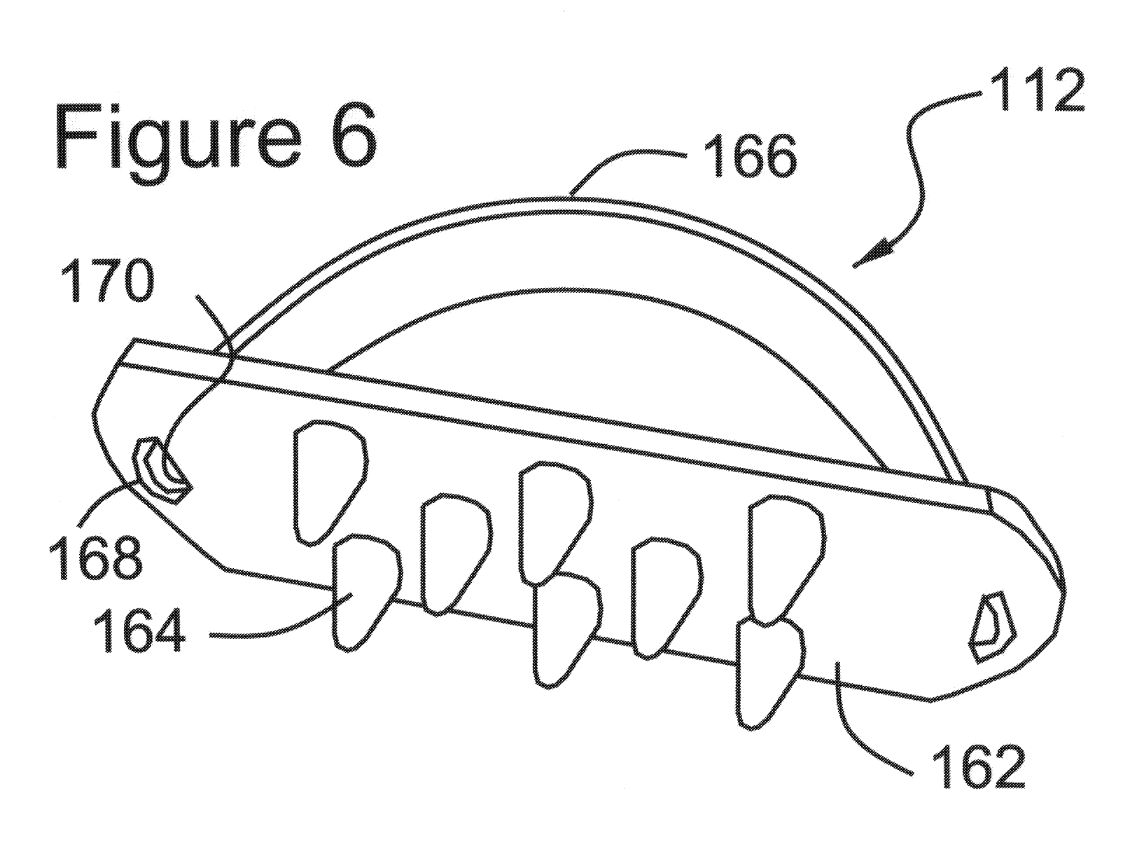

As aforementioned, the apparatus formed by the two compression components resembles a cuff, as illustrated in FIGS. 1 and 6. When the user desires to treat an affected area, the user surrounds the affected area of his/her arm with armband 10, for example in the configuration illustrated in FIG. 4. One then tightens main band 38 by bringing it from the position illustrated in FIG. 4 to the position illustrated in FIG. 1 following the movement indicated by arrow 56, as illustrated in FIG. 3. The band is then secured using hook Velcro member 50. The result is to secure armband 10 to the arm, ensuring that elastic member 34 remains directly over the affected area.

Once the armband is secured around the affected area one may change or insert the treatment device 12 while the armband 10 is in its fastened state, as illustrated in FIG. 5. This is done by detaching pressure-applying band section 20, as illustrated in FIG. 5, and inserting the desired treatment device, such as a hot pack, cold pack or acupuncture device into the pocket provided by tab 32. In this state the user inserts either of the various treatment modality elements 12 (including, but not limited to, heat therapy, cold therapy or acupressure therapy) into the pocket 58. Upon insertion of a treatment modality, the user tightens section 20 in the position illustrated in FIG. 1 by securing fastening element 28 in the position illustrated in FIG. 1. Once fastened the modality treatment device is nestled on the breathable, expandable compression platform formed by elastic members 20 and 34, thereby creating exposure of treatment device 12 to the affected area of the user's arm.

In accordance with the invention it is contemplated that the treatment area will be in between the elbow and wrist on the inside of the arm in between the elbow 60 and inside elbow (antecubital fossa), as illustrated in FIGS. 5, 7 and 9, in which the elbow 60 is shown in dashed lines.

The inventive armband has a number of advantages. In particular, it removes the difficulties of mounting and fastening armband 10. Additionally, it contains the disclosed pocket or open tab 32 which facilitates the mounting of various treatment devices 12 tailored for receiving various treatment modalities, including heat therapy, cold therapy and acupressure therapy, which increase blood flow and accelerate the healing of the epicondyle tendon. Moreover, it contains a breathable, expandable compression platform, created by elastic members 20 and 34, which facilitates temperature transfer to the effected area of the user's arm optimizing the therapeutic effect of the inventive armband.

In the present specification, several embodiments of the invention are illustrated. Parts having similar or analogous functions in the various embodiments are numbered with numbers which are multiples of 100 different from the numbers associated with corresponding elements in FIG. 1, to the extent possible or practical.

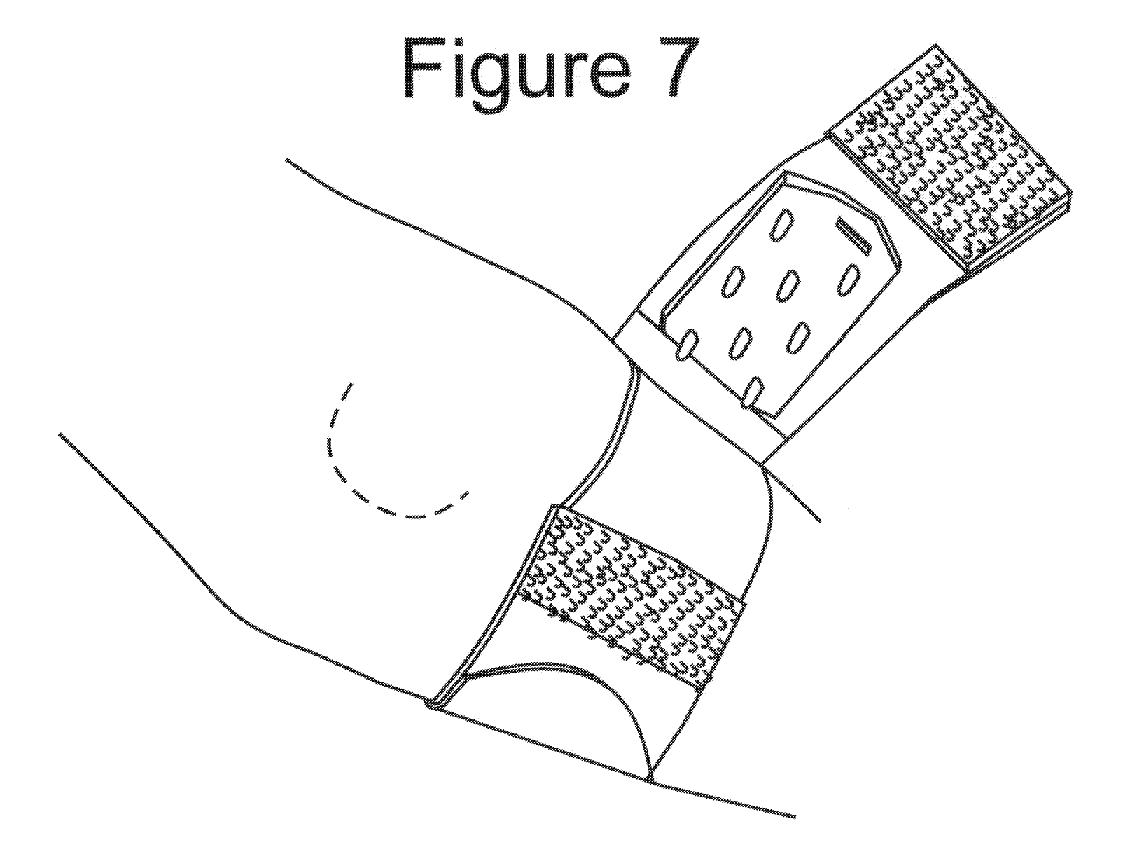

As shown in FIG. 6, heat pack 12 may be replaced by a spike plate 112 for acupressure therapy. Spike plate 112 comprises a face 162 having a plurality of spikes 164 formed integrally therewith as a single piece. Spikes 164 have a height of approximately 8 mm and a radius at the point of approximately 2 mm, although dimensions of other spike plates commonly available on the market may be used depending upon user preference or experienced effectiveness. The spike plate may be used with a handle or finger strap 166, which is secured by engagement structures 168. Finger strap 166 is secured by engagement structures 168 after latching into associated holes 170, of conventional design. With finger strap 166, spike plate may be grasped to apply pressure manually. Finger strap 166 may also be removed to allow placement in the inventive armband. Spike plate 112 is positioned in the manner of heat transfer pad 12, as illustrated in FIG. 7.

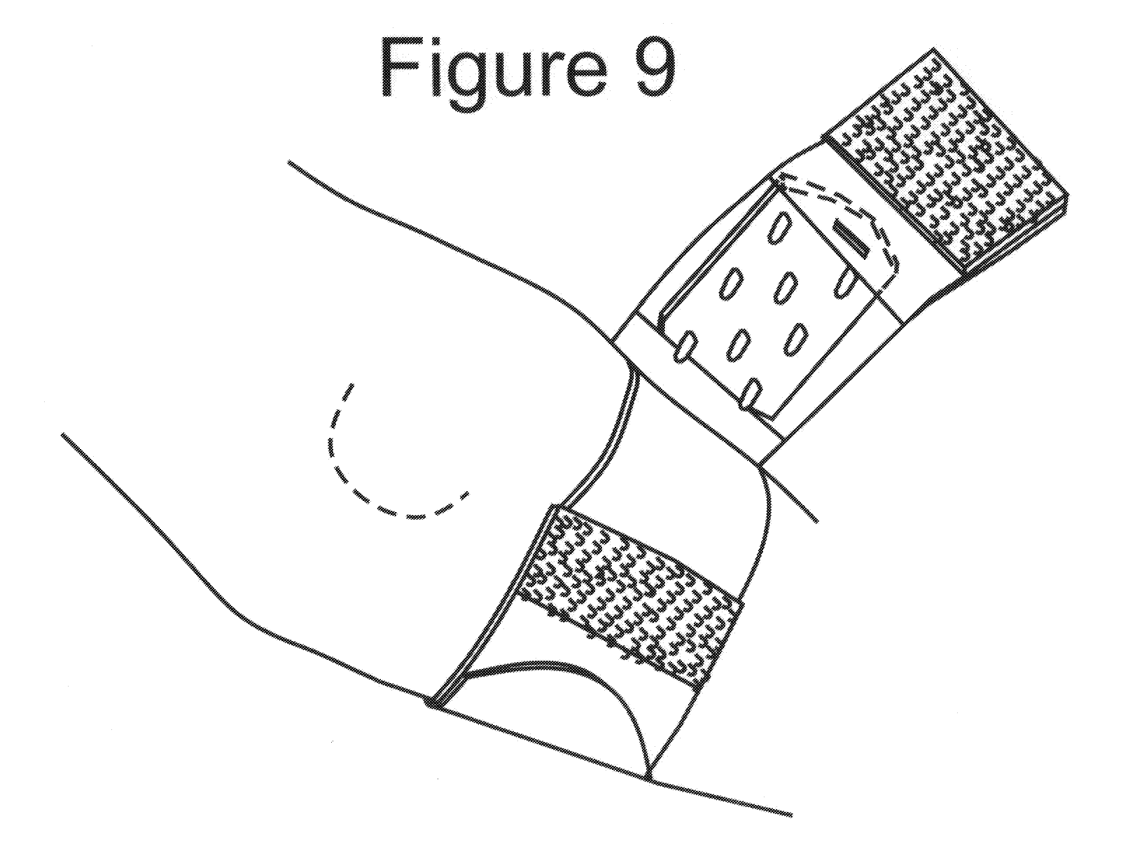

Referring to FIG. 8, an embodiment of the invention comprising an armband 210, in which the treatment device, such as a hot pack, cold pack or spike plate 212 is held in position by two full pockets formed by stitches 272. Spike plate 212 is securely held in position, as illustrated in FIG. 9. This allows fastening element 228 to be easily adjusted as desired without fear of treatment device 212 coming loose and falling.

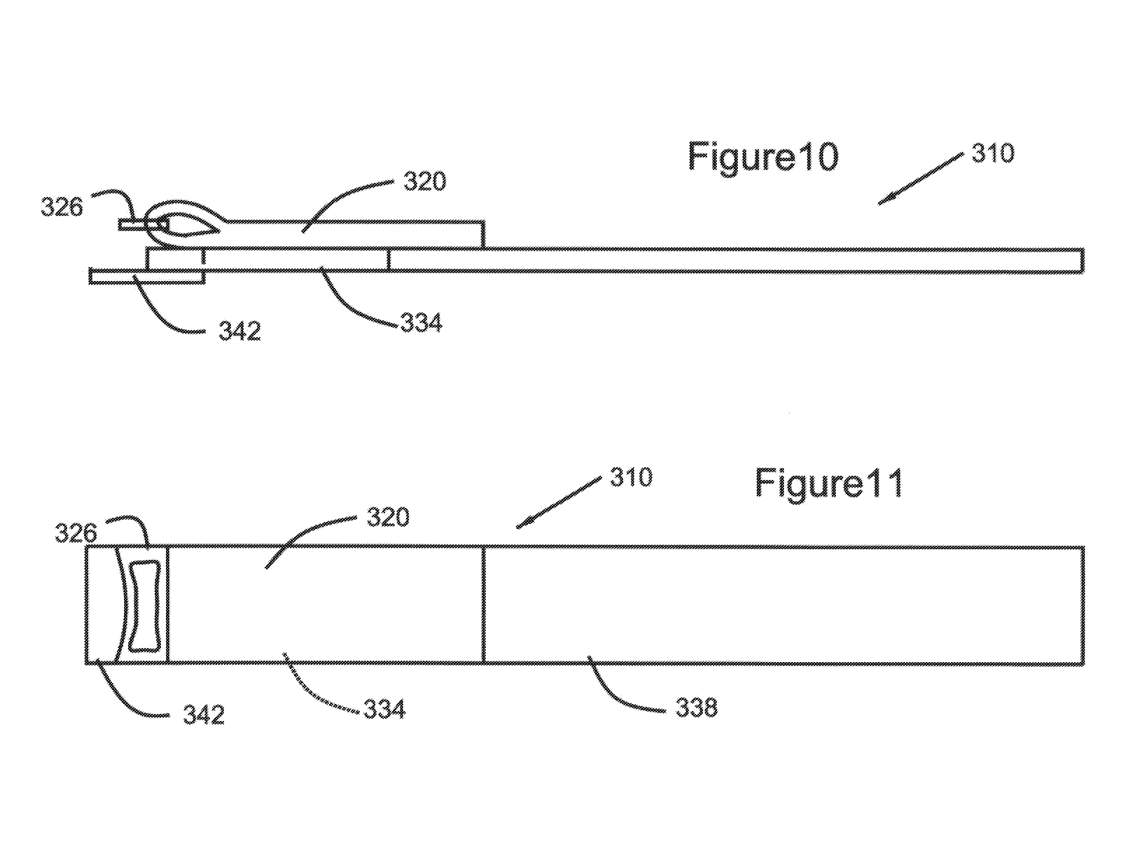

A schematic diagram of a preferred embodiment of the inventive armband is illustrated in FIG. 10. In this embodiment, rectangular frame 326 has a length of approximately 2 cm. Shield 242 has a length of approximately 5 cm and overlaps elastic member 334 over length of 2.5 cm. Elastic member 334 has an elastic length of approximately 9 cm. Main band member 338 has a length of approximately 26 cm., as illustrated substantially to scale. Finally, the width of armband 310 is approximately 5 cm.

In accordance with the invention, it is contemplated that in addition to the application of heat, and/or cold, as well as the application of acupressure through the use of the spike plate, the performance of appropriate exercise during wearing of the inventive armband will enhance effectiveness. This is especially the case when using the spike plate in the inventive armband. Applicant has discovered that particular effectiveness is achieved when using the inventive armband, with the spike plate installed, in combination with a specialized device for exercising muscles particularly impacted by epicondylitis.

More particularly, it has been found that wearing the inventive armband, with the spike plate installed in the armband applying acupressure, the exercise provided by hand expansion/hand contraction exercise provided by using the device (now commercially available and sold under the trademark Extensor) illustrated in U.S. Pat. No. 7,740,561 (the disclosure of which is hereby incorporated herein by reference) is particularly effective. Substantial improvement of the condition may be achieved by exercising as little as, for example, 90 seconds, opening and closing the fist every 2 to 4 seconds.

While illustrative embodiments of the invention have been described, it is noted that various modifications will be apparent to those of ordinary skill in the art in view of the above description and drawings. Such modifications are within the scope of the invention which is limited and defined only by the following claims.

What is claimed:

1. A method of treating epicondylitis, comprising applying to the elbow of a user suffering from epicondylitis a comprehensive treatment system that integrates various treatment modalities onto one platform.

2. A method of treating epicondylitis, comprising the therapeutic treatment of an elbow suffering from epicondylitis by increasing blood flow to the epicondyle tendon using a comprehensive treatment system that integrates various treatment modalities.

3. An improved elbow armband comprising a dual compression system wherein said dual compression system being adapted to allow the integration of various treatment modalities onto said dual compression system.

4. Apparatus as in claim 3, comprising an interchangeable compression platform; wherein said interchangeable compression platform receives various treatment modalities.

5. Apparatus as in claim 3, comprising an easy smart tab; wherein said easy smart tab prevents the dual compression system from becoming undone and facilitates mounting and fastening.

6. Apparatus as in claim 3, comprising a breathable, expandable compression platform, wherein said interchangeable compression platform is juxtaposed on the limb upon fastening of the dual compression system.

7. Apparatus for applying pressure, heat, cold or accu-pressure to a limb, comprising:
(a) band members, at least a portion of which are elastic;
(b) a tab element, at least a portion of which is stiff and plastic, forming a catch for receiving and holding various treatment modalities;
(c) a shield, at least a portion of which is thin, flexible and smooth, for preventing the user from becoming uncomfortable during treatment;
(d) a safety tab, at least a portion of which is stiff and plastic, for preventing the apparatus from coming undone, and thereby facilitating securement of the armband around the affected area;
(e) a pocket element for receiving various treatment modalities;
(f) a rectangular frame, at least a portion of which is stiff and plastic, for connecting band members and forming a cuff-like apparatus.

8. Apparatus as in claim 7, wherein the band members contain an elastic member of which is sufficiently in contact with the user's limb to facilitate temperature transfer and acupressure.

9. Apparatus as in claim 7, wherein the band members are comprised of an elastic pressure-applying band section which is folded upon itself at one end by stitches, to form a loop enclosing one side of a rectangular frame at one end and at the other end, a plain fabric fastening element carrying a male hook fastening element secured by stitches.

10. Apparatus as in claim 7, wherein the band members are comprised of a main band member which holds a female hook fastening element, which mates with male hooks on the elastic pressure-applying band section, and forms a compartment holding treatment modalities.

11. Apparatus as in claim 7, wherein the band members are comprised of a main band member that is secured by stitches to an adjustable securement member, said adjustable securement member may be positioned as desired to achieve the desired degree of pressure.

12. Apparatus as in claim 11, wherein said adjustable securement member is secured at one end to a male hook member and a safety tab by stitches, said male hook member being secured at the other end by stitches to the safety tab.

13. Apparatus as in claim 11, wherein said adjustable securement member is wrapped around a frame member and adjusted to the length or pressure desired and secured to by a male hook member to female hook member, resulting in the formation of a cuff-like armband.

14. Apparatus as in claim 7, wherein said band members are comprised of an elastic member and an elastic pressure-applying band section, creating an expandable compression platform which can accommodate treatment elements of various sizes.

15. Apparatus as in claim 9, wherein said elastic pressure-applying band section comprises a structure providing for adjusting a treatment modality around the affected area of the user's arm.

16. Apparatus as in claim 9, wherein said elastic pressure-applying band section 20 comprises an open tab, wherein said open tab creates a pocket capable of fitting and receiving various treatment modalities.
